(19) United States
(12) Plant Patent Application Publication
(10) Pub. No.: US 2006/0143756 P1
(43) Pub. Date: Jun. 29, 2006

(54) CHRYSANTHEMUM PLANT NAMED 'HOMER'

(50) CHRYSANTHEMUM MORIFOLIUM; CHRYSANTHEMUM

(75) Inventor: Mark Roland Boeder, The Hague (NL)

Correspondence Address: Charles A. Wendel;STEPTOE & JOHNSON LLP, 1330 Connecticut Avenue, N. W., Washington, DC, 20036, US

(73) Assignee: Chrysanthemum Breeders Association N.V., Aalsmeer (NL)

(21) Appl. No.: 11/020,258

(22) Filed: Dec. 27, 2004

(51) Int. Cl. A01H 5/00

(52) U.S. Cl. PLT/290

(57) ABSTRACT

A chrysanthemum plant named ‘Homer’ characterized by its medium sized blooms with orange ray florets and good branching; natural season flower date August 16-23; blooming for a period of 6 weeks.

### BACKGROUND OF THE INVENTION

‘Homer’ is a product of a breeding and selection program for outdoor pot mums (garden mums) which had the objective of creating new chrysanthemum cultivars with a decorative type flower, a natural season flower date around August 16-23; blooming for a period of 6 weeks. The new plant of the present invention comprises a new and distinct cultivar of Chrysanthemum plant ‘Homer’ is is a seedling resulting from a crossing program, which was set up by a previous breeder, and which records are unknown to the inventor. The new and distinct cultivar was discovered and selected as a flowering plant by Mark Roland Boeder on a cultivated field in Rijsenhout, The Netherlans in 2001. The first act of asexual production of ‘Homer’ was accomplished when vegetative cuttings were taken from the initial selection in 2001 in a controlled environment in Rijsenhout, The Netherlans, and propagated further at this location. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

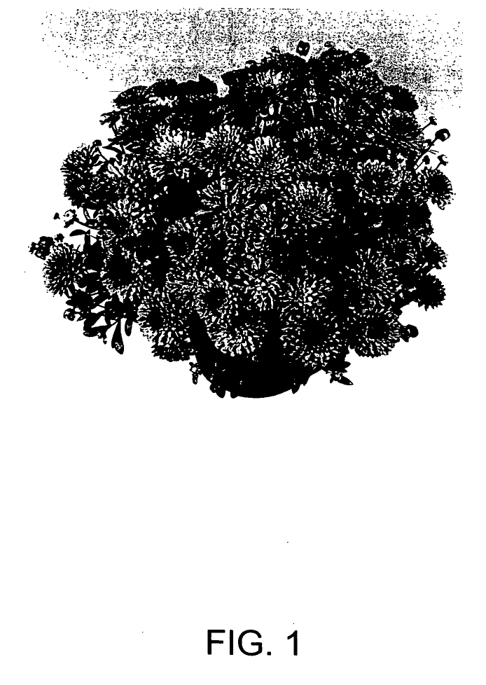

FIG. 2 shows the various stages of bloom of the new cultivar.

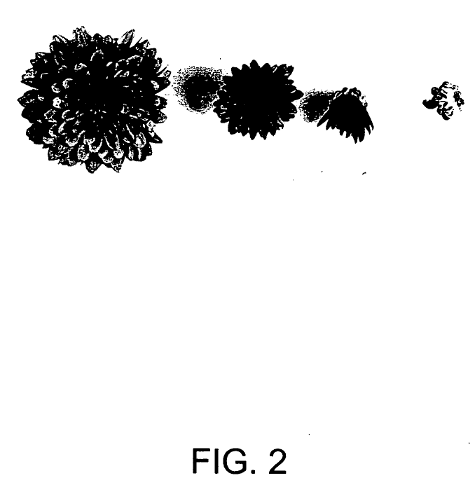

FIG. 3 shows the foliage of the new cultivar.

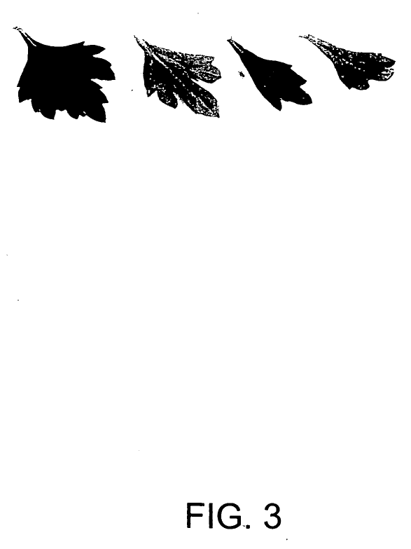

### DESCRIPTION OF THE INVENTION

This new variety of chrysanthemum is of the botanical classification Chrysanthemum morifolium L. The observations and measurements were gathered from plants grown out door in Rijsenhout, The Netherlans under natural day length and temperature and planted week 24 in 2004. The natural blooming date of this crop was August 16-23 (week 34). The average height of the plants was 25 cms. No growth retardants were used. No tests were done on disease or insect resistance or susceptibility. No tests were done on cold or drought tolerance. This new variety produces medium sized orange blooms flowering for a period of 6 weeks.

From the cultivars known to inventor the most similar existing cultivars in comparison to ‘Homer’ are ‘Beryl’ (U.S. Plant Patent pending) and ‘Hector’ (U.S. Plant Patent pending). When ‘Homer’, ‘Beryl’ and ‘Hector’ are being compared the following similarities and differences are noticed: All three varieties have medium sized decorative type orange blooms. The differences of ‘Homer’ and ‘Beryl’ and ‘Hector’ are (1) Number of ray-florets. ‘Homer’ has a higher number of ray-florets than ‘Beryl’ and ‘Hector’. (2) Size of bloom. Because of this high number in ‘Homer’, its blooms are large and more spherical shaped than those in ‘Beryl’ and ‘Hector’.

The following is a description of the plant and characteristics that distinguish ‘Homer’ as a new and distinct variety. The color designations are taken from the plant itself. Accordingly, any discrepancies between the color designations and the colors depicted in the photographs are due to photographic tolerances. The color chart used in this description is: The Royal Horticultural Society Colour Chart, edition 1995.

TABLE 1
Botanical Description of chrysanthemum plant ‘HOMER’

| | |
|---|---|
| Bud | |
| Size | Small; cross-section 0.5 cm, height |
| | 0.5 cm |
| Outside Color | Yellow-green 145D |
| Involucral bracts | 2 rows, length 7 mm, width 3 mm |
| Involucral bracts among disc-florets | Not present |
| Involucral bracts color | Green 138A-138B |
| Bloom | |
| Type | Decorative |
| Height | 2 cm |
| Size | Medium |
| Fully Expanded | 5.5 cm |
| Peduncle length | 6-7 cm |
| Peduncle color | Green 139C |
| Number of blooms per branch | Approx. 6 blooms per branch |
| Performance on the plant | 6 weeks |
| Seeds | Produced in small quantities, ovate |
| | grey-brown 199A, 1½ mm in |
| | length. |
| Fragrance | Typical chrysanthemum, slightly |
| Color | |
| Center of the flower | Immature Greyed-orange 171B |
| | Mature Greyed-orange 168B |
| Color of upper surface of the ray- | Yellow-orange 17D overlain with |
| florets | Greyed-orange 167C |
| Color of the lower surface of the | Greyed-orange 164C overlain with |
| ray-florets | Greyed- orange 167C |
| Tonality from Distance | A garden mum with orange flowers |
| Discoloration to color | Greyed-yellow 162B with |
| | Greyed-orange 164B |
| Ray florets | |
| Texture | Upper and under side smooth |
| Number | 280-300 |
| Cross-section | Flat |
| Longitudinal axis of majority | Straight |
| Length of corolla tube | 0.5 cm |
| Ray-floret margin | Entire |
| Ray-floret length | 2.5 cm |
| Ray-floret width | 0.6 cm |
| Ratio length/width | Medium |
| Shape of tip | Dentate |
| Disc florets | Absent |
| Receptacle shape | Conical raised |
| Reproductive Organs | |
| Stamen | Absent |
| Styles | Thick |
| Style color | Yellow 13A |
| Style Length | 4 mm |
| Stigma color | Yellow-green 144A |
| Stigma Width | 1 mm |
| Ovaries | Enclosed in calyx |
| Plant | |
| Form | Grown as a spray type potmum, |
| | outdoor mounded and round |
| Growth habit | Spherical and spreading |
| Growth rate | Medium |
| Height | 30 cm |
| Width | 35 cm |
| Stem Color | Green 138B with streaks of |
| | Greyed-red 182B |
| Stem Strength | Medium |
| Stem Brittleness | Brittle |
| Stem Anthocyanin Coloration | Present |
| Internode length | 2-2 5. cm |
| Length of lateral branch | From top to bottom 13 cm |
| Lateral branch color | Green 138B-138C |
| Lateral branch, attachment | Weak |
| Branching (average number of | Good with 8-10 breaks after |
| lateral branches) | pinching |
| Natural season blooming date | August 16-23 |
| Foliage | |
| Leaf color | Upper side Green 138A |
| | Lower side Green 138B |
| Color midvein | Upper side Green 139D |
| | Lower side Yellow-green 148D |
| Size | Medium; length 4-6 cm, width |
| | 2.5-4 cm |
| Quantity (number per lateral | 20 |
| branch) | |
| Shape | Obovate |
| Texture upper side | Glabrous |
| Texture under side | Pubescent |
| Venation arrangement | Palmate |
| Shape of the margin | Serrated |
| Shape of Base of Sinus Between | Rounded |
| Lateral Lobes | |
| Margin of Sinus Between Lateral | Diverging |
| Lobes | |
| Shape of Base | Acute-obtuse |
| Apex | Mucronulate |
| Petiole lenght | 1-2 cm |
| Petiole color | Green 139D |

TABLE 2
Differences with the comparison varieties

| | ‘Homer’ | ‘Beryl’ | ‘Hector’ |
|---|---|---|---|
| Bloom type | Decorative | Decorative | Decorative |
| Color upper side | Yellow-orange 17D and | Orange | Orange- |
| ray-florets | Greyed-orange 167C | 26C | red 34C |
| Number of ray-florets | 280-300 | 250 | 250 |
| Size of bloom | 5.5 cm | 4.5 cm | 5.5. cm |

I claim:

1. A new and distinct variety of chrysanthemum plant as described and illustrated.
